(19) United States
(12) Patent Application Publication
(10) Pub. No.: US 2024/0293196 A1
(43) Pub. Date: Sep. 5, 2024

(54) MEDICAL REMOTE CONTROL, CONTROL SYSTEM, AND CONTROL METHOD

(71) Applicant: B. Braun New Ventures GmbH, Freiburg im Breisgau (DE)

(72) Inventors: Sebastian Zepf, Wurmlingen (DE); David Hofmann, Freiburg (DE)

(21) Appl. No.: 18/575,667

(22) PCT Filed: Jun. 28, 2022
(86) PCT No.: PCT/EP2022/067774

§ 371 Date: Dec. 29, 2023

(30) Foreign Application Priority Data
Jun. 30, 2021 (DE) 10 2021 116 915.1

(51) Int. Cl. A61B 34/35; A61B 17/00; A61B 34/00; A61B 34/30

(52) U.S. Cl. CPC A61B 34/35; A61B 17/00; A61B 34/74; A61B 2017/00212; A61B 2017/00221; A61B 2017/00389; A61B 2034/305

(57) ABSTRACT

A medical remote control for wirelessly controlling a medical instrument or device includes a housing having a fastening portion to be fastened to an object, in particular the medical, digitally controllable instrument or device; an actuating button located in or on the housing, in particular exactly one single actuating button, for generating an input signal by manual actuation of the actuating button; a wireless data interface for establishing a wireless data connection to an external data interface, in particular a data interface of the medical instrument or device, and for transmitting data; and a control unit for processing the input signal of the actuating button and determining an associated control signal based on the input signal and to transmit the control signal via the data interface. The medical remote control can be used with or in a control system, a control method, and a computer-readable storage medium.

### CROSS-REFERENCE TO RELATED APPLICATIONS

This application is the United States national stage entry of International Application No. PCT/EP2022/067774, filed on Jun. 28, 2022, and claims priority to German Application No. 10 2021 116 915.1, filed on Jun. 30, 2021. The contents of International Application No. PCT/EP2022/067774 and German Application No. 10 2021 116 915.1 are incorporated by reference herein in their entireties.

### FIELD

The present disclosure relates to a medical remote control/remote operator for wirelessly controlling at least one medical, in particular surgical, instrument or a medical (digitally controllable) device, such as a surgical robot. In addition, the disclosure relates to a medical control system/controlling system, a medical control method and a computer-readable storage medium according to the preamble of the independent claims.

### BACKGROUND

Nowadays, digital or digitally controllable surgical instruments or devices often require remote control by a surgeon from a sterile field near the surgical site. However, the surgeon usually needs both hands simultaneously to operate currently used surgical tools.

Acoustic remote control of the instrument or device via speech and speech recognition, which issues a corresponding control command or control signal, currently still suffers from a lack of robustness, since the voice command is not reliably and unambiguously recognized and converted into a control command due to different pronunciations, a face mask, or other background noise. Voice control therefore usually fails due to the need to ensure patient safety. In addition, if the surgeon's concentration is too low or an unintentional statement is made, a control command may be triggered unintentionally.

However, if keys or a touch display from a separate station, such as a tower, are used for remote control, these manual inputs almost always require the surgeon to interrupt the surgery in order to perform the corresponding remote control.

However, if remote controls are integrated directly into surgical instruments, this integration is often associated with compromises in terms of the ergonomics of the instrument and involves a great deal of effort in the corresponding technical development. It is also currently the case that if several alternative functions are to be activated with the remote control, a plurality of physical keys are usually implemented. In addition, such a remote operator is not interchangeable in the event of further technical development.

U.S. Pat. No. 8,939,891 B2, for example, discloses a medical robotic system with a medical instrument and an input device with which a surgeon can control the medical instrument. However, the input device is very complex and a surgeon can hardly switch between the input device and the actual surgical field.

### SUMMARY

The objects and objects of the present disclosure are therefore to avoid or at least reduce the disadvantages of the prior art and in particular to provide a medical remote control, a medical control system, a medical control method, and a computer-readable storage medium which allows control of a medical instrument or device in a particularly simple and intuitive manner and in particular is compact and can be flexibly mounted in the surgical environment. A further partial object is to provide a universal remote operator with which different medical instruments and devices can be controlled. A further partial object is to provide a practicable and sterilizable remote control and control system that hardly interferes with the usual working environment when used in a sterile room.

These objects are solved with respect to a medical remote control by the features of claim 1, with respect to a medical control system by the features of claim 10, with respect to a medical control method by the features of claim 14 and with respect to a computer-readable storage medium by the features of claim 15.

A basic idea of the medical remote control is therefore to provide a remote control with an actuating button/an (actuation) key, in particular in the form of a single key, with a wireless communication interface to control an external device with a corresponding external communication interface/user interface. In particular, a standardized communication interface with a standardized protocol is used to make the remote operator compatible with a plurality of instruments and devices for universal use. A specially adapted control unit processes the actuation of the actuating button and outputs a corresponding control signal directly or indirectly to the medical instrument or device. The medical instrument or device, which is digitally controllable, can then perform a corresponding control based on the control signal. The surgeon can thus perform remote control via a haptic movement of the actuating button. In particular, the special remote control with specially configured control unit makes it possible to activate several different events or send several different control signals with the actuating button on the basis of different input signals.

The flexibly usable medical remote control therefore has an actuating button and can be positioned at any site in the sterile field. It gives the surgeon the option of positioning the remote control at a desired location. In particular, the remote control is configured as a flexible one-button remote control (flex button) and only has a single actuating button. In this way, the installation space can be kept even smaller and the remote control can be configured even more simply and can be attached more flexibly to objects. For example, the remote control may be attached to a surgical instrument such as a suction tube in the field of neurosurgery or to a drape near the patient or to an imaging device (surgical monitor) or to a surgical robot via its fastening portion. The remote control/remote operator communicates wirelessly and can communicate via Bluetooth in particular. The remote control can communicate with any medical instrument or device that has a suitable data interface, such as Bluetooth, and can control this instrument or device remotely. In particular, standardized control signals are also used.

The input signal can be processed directly in the control unit of the remote control. In particular, a corresponding control signal can be determined based on various key pressing patterns as the input signal.

In other words, a remote control (and control system) is proposed comprising an electronic key, in particular single key, with a wireless communication interface, wherein the instrument or device is controlled with a wireless user interface. A specially adapted control unit receives various key pressing patterns as an input signal and is adapted to determine and transmit a control signal (from various remote control modes) based on this key pressing pattern. A portion, in particular a rear side, of a remote control housing is in particular provided with an adhesive for attaching the housing to any site in the sterile field so that a surgeon can perform complex remote control of electronic devices from the sterile field.

In yet other words, the disclosure relates to a medical remote control for wirelessly controlling a medical, in particular surgical, instrument or a medical electronic device, comprising a housing having a fastening portion for, in particular detachably, fastening the housing to an object separate from the remote control; an actuating button arranged in or on the housing, in particular exactly one actuating button, for generating an input signal via manual actuation of the actuating button; a wireless data interface for establishing a wireless data connection to a suitable external data interface, in particular a data interface of a medical instrument or device, and for transmitting data, and a control unit which is specially provided and adapted to process the provided input signal of the actuating button and to determine an associated control signal on the basis of the input signal and to transmit this particular control signal as a control command via the data interface. Due to its small and compact design, such a remote control can be flexibly installed anywhere in the sterile area and can be adapted to the individual needs of the surgeon. For example, the remote control can be repositioned at different operating times with different surgery modalities in order to always achieve the best possible integration of the remote control/remote operator into the overall surgery.

The term ‘control signal’ defines a control signal stored in the remote operator, which can be processed and executed by a medical instrument or device. For example, this control signal may be ‘suction on’ of an aspirator, which the aspirator converts accordingly.

Any disclosure relating to the medical remote operator also applies to the medical control system and the (control) method, just as any disclosure relating to the control system and the method also applies to the remote operator. The features of remote operator, control system, and control method are interchangeable.

Advantageous embodiments are claimed in the dependent claims and are explained in particular below.

According to one aspect of the present disclosure, the control unit may be adapted to detect a temporal length/duration and/or a number of actuations within a defined time interval of the actuating button as an input signal and to transmit the corresponding control signal based on this, in particular based on an assignment of input signal to control signal stored in a storage unit. The control unit therefore detects a type of pressing pattern as an input signal when the actuating button is actuated and determines an associated control signal using the pressing pattern on the basis of a stored assignment. In particular, the remote control or the control unit can distinguish between short pressing (single click), long pressing (within a time interval), double pressing (double click) and a continuous pressing in order to activate various command functions. By distinguishing, for example, between short pressing, long pressing, double pressing and continuous pressing, a user can switch back and forth between functions.

Preferably, the control unit and/or the remote operator may have a storage unit in which a current control state of the remote control is stored as a parameter and the control unit may be adapted to finally determine the control signal on the basis of the current control state on the one hand and the input signal on the other. In particular, a control diagram or a table with a first column of the current control state, a second column of the input signal and a third column of the associated control signal may be stored in the control unit, so that the appropriate combination of the two parameters generates a unique control signal which is output to the medical instrument or device. The control signal is determined on the basis of these two parameters. For example, in order to move a robotic microscope in any direction, a double press/double click switches between ‘left’ and ‘right’, ‘upward’ and ‘downward’ and ‘forward’ and ‘backward’. In particular, the control unit may therefore be adapted to click/cycle through the directions (of the coordinate system used) one after the other in the case of a single click and to reverse the direction (opposite direction) in the case of a double click. Alternatively, in one embodiment it may be provided that a single click reverses the direction (i.e. from ‘upward’ to ‘downward’) while a double click alternates between translation along an axis and rotation around an axis.

According to one aspect of the disclosure, the storage unit may be provided centrally, in particular as a cloud storage (computer cloud/data cloud) and/or as a storage unit of a server and/or as a storage unit of the medical instrument or device, and the control unit can, in particular via the wireless data interface, access the data of the central storage unit, can in particular send and receive data. Since the storage unit is provided centrally that means that data can be stored and manipulated centrally. In particular, data consistency is ensured. Replacing a medical remote operator is also simplified, since the important data is available centrally and no data transfer from one remote operator to the next remote operator is necessary in the event of a change. Several remote operators may also be used to control a single instrument or device. In addition, data maintenance and troubleshooting can be carried out more easily.

According to a further aspect of the present disclosure, a relation or a flowchart of several control states can be stored in the storage unit and the control unit may be adapted to proceed to a following, different control state corresponding to the relation of the control states on the basis of this relation of the control states and an input signal selected for this relation. For example, in the case of the remote control, which is connected to a robot arm via the data interface, a control state may be: ‘move robot arm translationally upward’. If the actuating button is pressed twice (double click as input signal), the control unit accesses the stored relation of the control states and determines on the basis of the control state and the double click that the new, current control state should be ‘move robot arm translationally downward’ (direction reversal). If, for example, the actuating button is pressed for longer than 1 second, the control unit determines on the basis of the control state ‘move robot arm translationally downward’ that the control signal should be ‘move robot arm translationally downward’ and thus controls the robot arm downward. A single key can be used to switch between control states (‘toggling’) and the corresponding command can then be selected in the state and sent as a control signal.

According to one embodiment, the remote control may have a display. Preferably, the control unit may be adapted to show the current control state of the remote control on the display. In this way, the surgeon or a user of the remote control always has the current control state in view.

According to a further aspect of the disclosure, the actuating button may be adapted to have, in addition to a translational, rectilinear/linear degree of freedom (a rectilinear/linear movement upward and downward along a longitudinal axis of the actuating button), a degree of freedom about at least one pivot axis perpendicular to the longitudinal axis of the actuating button, in particular at least two pivot axes for four pivot directions. Alternatively or additionally, the actuating button may have a degree of freedom of rotation about the longitudinal axis of the actuating button in order to increase the number of actuation alternatives and provide input alternatives. The key can be pressed with a tilt, so to speak, to enable two or more commands. In particular, the actuating button may be configured as a tilt button and rotate button and push button, which allows movement in six directions and is also rotatable about its longitudinal axis. The longitudinal axis is in particular parallel to the direction in which the manual pressure is applied to the actuating button. Typically, this longitudinal direction is perpendicular to the outer surface of the actuating button.

According to a further embodiment, the fastening portion may have an adhesive surface, in particular an adhesive side, in order to attach/detachably fasten the remote control to an external object. In this way, a temporary, firmly bonded connection can be created on the back of the remote control and quick attachment to the desired location is made possible. In particular, the adhesive side is sterile so that the remote control can be placed in the sterile area.

According to a further aspect of the disclosure, the remote control may be configured to be sterile or sterilizable. In particular, it has a fluid-tight sheathing/sheath made of sterilizable materials in order to be sterile or sterilizable. In particular, the remote control itself is sterilely packaged so that the packaging can only be opened and safely used at the place of use. The remote control is therefore configured to be sterile or sterilizable in particular, either for single use or for reprocessing.

According to a preferred embodiment, the medical remote control may further comprise an inertial sensor/inertial measurement unit (IMU) to detect a movement and the control unit may be adapted to determine a corresponding control signal based on at least the input signal and the movement. In particular, the detected movement may be correlated with a movement control signal. If, for example, the instrument or device to be controlled is to execute a movement, the inertial measurement unit can be used to translate a movement of the remote control into a corresponding control signal. If the remote control moves upward, a robot arm moves upward, for example.

In particular, the remote control, preferably the housing, may be adapted to be worn on a person's wrist. Further preferably, the fastening portion may be configured as a bracelet, similar to a watch strap, in order to make the remote control quickly detachable.

In particular, the data interface has a WLAN interface and/or a Bluetooth interface. These two wireless data transmission formats are standardized, secure and compatible with most medical instruments and devices.

In particular, the actuating button may be adapted to detect the intensity of the pressure and forward it as an input signal. In this way, for example, the intensity/speed of a movement can be correlated with the intensity of the detected pressure.

Preferably, the remote control may be configured as a sterile disposable/single-use product.

According to a further aspect of the disclosure, the remote control may have an energy storage device in the form of a rechargeable battery, in particular a Li-ion battery. In particular, the remote control and the rechargeable battery may be adapted to be charged inductively without contact. Thus, the remote control is self-sufficient.

With regard to the medical control system for controlling a medical, in particular surgical, instrument or a medical, digitally controllable device, the objects are solved in that it comprises: at least one controllable medical instrument or device which is digitally controllable and has an external data interface; and at least one medical remote control according to the present disclosure. The data interface of the remote operator is compatible with the data interface of the medical instrument or device. In particular, the control signals associated with the instrument or device are compatible in the remote control. The input signal can be processed directly in the control unit of the remote control. Alternatively or additionally, the input signal can also be processed in an external control unit, such as a server in a surgical control system, and a corresponding control signal can be determined.

Preferably, the control system may comprise at least two, preferably at least three remote controls that control the at least one medical instrument or device. In particular, several remote controls may be provided and arranged (placed) in the operating field of the control system. These can, for example, allow the same or different control commands.

According to a preferred embodiment, the control system may further comprise a central server with a server control unit and the at least one controllable medical instrument or device and the at least one remote control may be connected to this server as a client or interact with the server. In this way, several remote controls can be integrated into the system, which can, for example, simultaneously control one medical instrument or device or even several. The server control unit can also process the control signals centrally on the server in such a way that the server control unit has the control state of the medical device stored and a final control signal can be determined on the basis of the control state and the control signal, which in this case corresponds to the input signal. In this way, a control state is stored centrally and the surgeon can switch between the individual remote controls without always having to change a control state again.

According to another aspect of the disclosure, the control system may comprise a surgical robot with a robot arm as a medical device, and the remote control may send a translational movement and/or a rotational movement of the robot arm as a control signal in order to control the robot arm.

As an alternative or in addition to a display of the remote control, the instrument or device may also have a display to show the current control state in particular. For example, a surgeon can simply look at a display, such as a surgical monitor of a surgical microscope, can read the control state there and can perform a haptic movement input of the actuating button for control according to this control state.

With regard to the medical control method for controlling a medical, in particular surgical, instrument or a medical controllable device via a remote control, the objects are solved by the steps of: establishing a wireless data connection between a medical remote control and a controllable medical instrument or device; detecting an input signal of the actuating button of the remote control, in particular an input pattern; determining, on the basis of the input signal, a control signal by the control unit; sending the determined control signal via the data interface of the remote control to the external data interface of the medical instrument or device.

With respect to a computer-readable storage medium, this comprises instructions which, when executed by a computer, cause the computer to perform the method/steps of the control method according to the present disclosure.

A medical sterile room may have the remote control or control system of the present disclosure.

In other words, according to the disclosure, a medical remote control is proposed in particular with in particular only a single, small and simple button that requires only a haptic movement from the surgeon. The specially adapted control unit (an implementation of a remote control software, so to speak) makes it possible in particular to activate (control) several different events with the actuating button/key. For example, these may be static events, such as function activation, or continuous events, such as movements of the instrument or device. In particular, the controlled device has a display that shows the current state (control state) of the remote operator (mode) (e.g. ‘move right’). When the desired mode is reached, the user can then execute the movement by, for example, continuously pressing a key. In particular, a translational and rotational movement of a device mounted on a robot arm can be performed with a single key of the remote operator. In particular, the remote control may be adapted to be attached to a surgical instrument, such as an endoscope, or to a housing of an electronic medical device, such as a surgical light or a base station of an endoscope, or to a surgical drape or to a surgeon's hand or arm, in order to provide it with flexibility on the desired object.

### BRIEF DESCRIPTION OF THE DRAWINGS

The present invention is explained in more detail below with reference to the accompanying Figures, with reference to preferred embodiments.

FIG. 1 shows a perspective view of a medical remote control of a preferred embodiment of a medical control system of a preferred embodiment,

FIG. 2 shows a partial perspective view of a remote control of a control system of a further preferred embodiment, which is attached to a robot arm,

FIG. 3 shows a schematic view of different actuation modes of the remote control, and

FIG. 4 shows a schematic view of a relation of control states, which change the control state via different press patterns,

FIG. 5 shows a perspective view of a remote control and a control system according to a further preferred embodiment, in which the remote control is attached to a housing of the OR cart with OR screen,

FIG. 6 shows a perspective view of a remote control of a preferred embodiment attached to a surgeon's arm, and

FIG. 7 shows a flow diagram of a control method according to a preferred embodiment.

The Figures are schematic in nature and are intended only to aid understanding of the disclosure. Identical elements are provided with the same reference signs. The features of the various embodiments may be interchanged.

### DETAILED DESCRIPTION

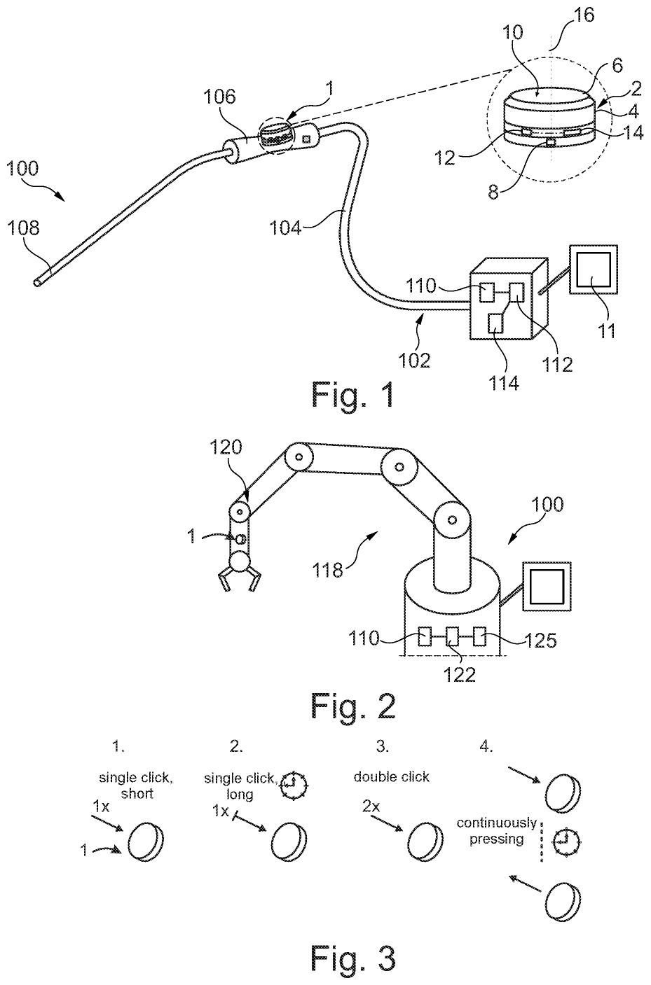

FIG. 1 shows a schematic, perspective view of a medical remote control 1 according to a first preferred embodiment in the form of a one-key remote control (flex button/flex key), which is used in a control system 100 of a preferred embodiment. Specifically, the remote control 1 for wireless control of a digitally controllable medical device has a housing 2 with a rigid, container-shaped housing base 4 with a hollow cylindrical (radial) outer wall and a flexible, upper housing cover 6, so that the remote control 1 is fluid-tightly sealed off from the environment.

The housing 2 itself has a fastening portion 8 on the underside of the housing base 4 in the form of a detachable adhesive surface/adhesive for attaching the remote operator 1 to various surfaces of external objects. A single actuating button/actuation key 10 is provided in the housing 2 itself or in the housing base 4, which is covered by the flexible housing cover 6 toward the front and is pressure-sensitive. In the event of manual actuation or haptic pressure being applied to the actuating button 10 by a surgeon, for example with the thumb, an input signal is generated and passed on to a control unit 12 in the remote control 1. This control unit 12 is specially adapted to process the input signal and to analyze it with regard to a pressing pattern/actuation pattern and to generate a corresponding control signal based on the actuation pattern. This control signal can then be sent wirelessly via a wireless data interface 14 of the remote control 1.

In addition to the remote operator 1, the control system 100 also has a medical instrument in the form of a medical aspirator 102 with a hose 104, a handle 106 and a distal suction tip 108. At the other end of the hose 104, the aspirator 102 has, at its proximal base, an external data interface 110, a suction control unit 112 and a pump 114 for actuation. The aspirator 102 is adapted by this configuration to receive a digital control signal and to perform a suction operation associated with the control signal based on the control signal. The wireless data interface 14 of the remote control 1 is wirelessly coupled to the external data interface 110, in this embodiment via a standardized Bluetooth connection, so that the control signal of the remote control 1 is transmitted to the control unit 112.

Specifically, a control state of a suction power from a set of ‘low’, ‘medium’ and ‘high’ of the aspirator 102 is stored in the suction control unit 112, wherein these three states can be selected alternately. If the surgeon now performs a double click as input (two manual actuations within a time interval, for example within one second), the control unit 12 processes this input and determines that a control state should be changed or progressed to the following control state (in this case from ‘low’ to ‘medium’, from ‘medium’ to ‘high’ or from ‘high’ to ‘low’). The current control state is shown on a display 116. If the user or surgeon presses the actuating button 10 for longer than a certain time, in this embodiment more than one second, the control unit 12 determines a control signal that the aspirator should suction with the current control state. The suction control unit therefore receives this control signal and actuates the pump 114 according to the power of the control state. If the surgeon releases the actuating button 10 again, the control signal also ends and the suction control unit 112 stops the pump 114.

The remote control 1 is particularly compact and handy due to its design and can be attached to different positions on the handle 106 depending on the preferences of the surgeon in charge. Due to its simple and cost-effective design, the remote control 1 may be configured as a disposable item and packed in sterile packaging. This increases patient safety.

The control system 100 has defined modes for the aspirator 102, and control signals associated with the input signals are stored in the control unit 12. This makes it particularly easy to couple the remote control 1 with the aspirator 102. In addition, further control signals for other medical devices can be stored in the control unit 12, so that several medical instruments and devices can be operated wirelessly with just a single remote control 1. In particular, the remote control 1 can perform an initialization process in order to be connected to just one medical device (for example, pressing the actuating button ten times in quick succession to start the connection/initialization process) and then confirm a connection to the desired medical device.

FIG. 2 shows a control system 100 according to a further embodiment in which, in contrast to the first embodiment, the medical device is a medical robot 118. The remote control 1 has the same structural design as the remote operator 1 of the first embodiment, but the control unit 12 is specially adapted to output a control signal that can be interpreted by the robot 118 when an input signal is received. In this embodiment, the remote control 1 is attached to the end effector or robot arm 120.

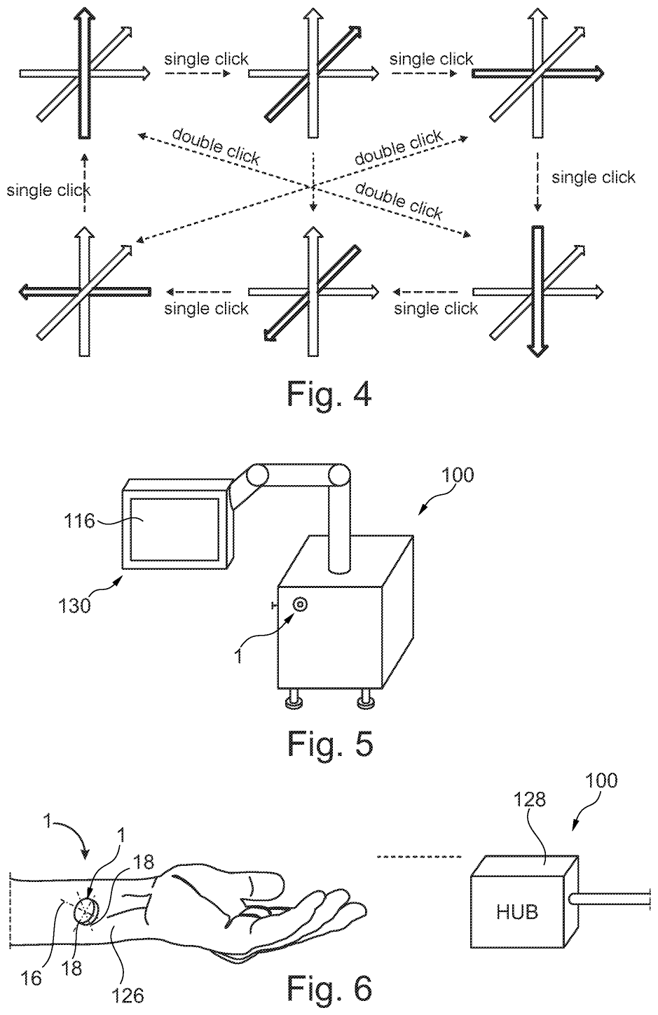

FIGS. 3 and 4 are used to explain how the control system in FIG. 2 works. 3 and 4 are used. FIG. 3 explains the different actuation patterns of the remote operator 1 of the control system 100. The single actuating button 10 can have different control functions and can in particular trigger a desired actuation of the robot 118. There is a (short) single click, which lasts less than one second, a (long) single click, which lasts longer than one second but less than two seconds, a double click of the actuating button 10, which is performed within one second, and continuous pressing, which may last any length of time over two seconds. An associated control signal is assigned to these four actuation modes as input signals by the control unit 12 in accordance with a control state.

The control states are explained below with reference to FIG. 4. In this embodiment, the control state of the robot 118 is stored in the control unit 12. According to FIG. 4, these control states may be a movement ‘upward’, ‘downward’, ‘forward’, ‘backward’, ‘left to side’ and ‘right to side’, corresponding to a Cartesian coordinate system provided at the base of the robot. A relation of the control states, as shown schematically in FIG. 4, is stored in the control unit 12. The thickly marked arrows represent the current control state.

Alternatively or additionally, this relation can also be stored in a storage unit 126 of a robot control unit 122. The display 116 shows this state accordingly (tool or robot arm ‘upward’). If the current control state is ‘upward’ and the surgeon continuously presses the actuating button 10, the robot arm 120 moves upward with the end effector driven by a motor. If a single click is now performed, the control unit 12 changes the control state to the following control state ‘backward’ based on the relation of the control states. If the actuating button 10 is now pressed again continuously, the robot 118 moves the end effector ‘backward’. In particular, the actuating button 10 is adapted to detect an applied pressure and the control unit is adapted to correlate a speed of movement with the detected pressure in the case of continuous pressure. This allows the surgeon to make a fine adjustment at low pressure and a broad adjustment of the end effector at high pressure.

If a double click is now performed in the ‘backward’ control state, the direction of movement changes to the opposite direction ‘forward’, and the control unit 12 uses the stored relation to determine that the new control state is ‘forward’. In this way, the direction of the three coordinate axes can be changed alternately with a single click, whereas a double click reverses the direction.

FIG. 5 shows a control system 100 of a further preferred embodiment, in which the remote operator 1 is attached to a medical cart 128 with a surgical monitor as a medical device. The remote operator can be used, for example, to change a display mode or to control a nearby medical device or instrument such as an endoscope.

FIG. 6 shows a schematic view of a control system 100 according to a further preferred embodiment. In this embodiment, the small remote control 1 with an adhesive back as a fastening portion is adapted to be adhered to an arm so that the surgeon always carries the remote control 1 with him/her. An external hub 128, which is provided on and connected to a medical instrument or device, receives the control signal and may also be adapted to transmit data to the remote operator 1. In this embodiment, the actuating button 10 is not only movable along its longitudinal axis 16, but also about two pivot axes 18 (all three axes 16, 18 are perpendicular to each other) in order to enable a tilting movement in addition to a pushing movement. For example, a control signal of a movement to the side can be interpreted during a tilting movement to the side.

The remote operator can therefore be installed wherever it is needed and connected to an Ethernet or hub via Bluetooth or similar wireless communication.

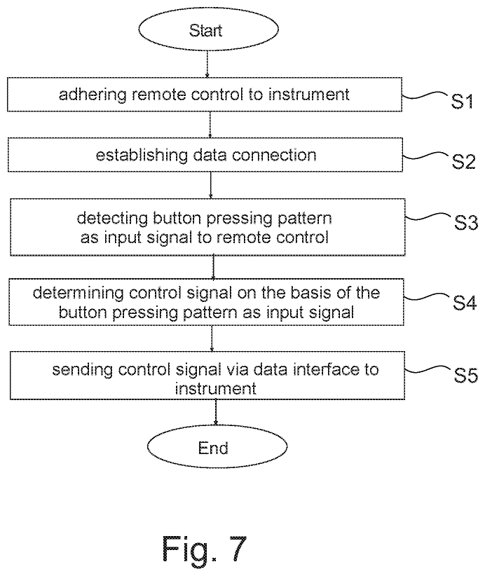

FIG. 7 shows a flow diagram of a control method according to a preferred embodiment.

In a first step S1, the remote control is attached to a medical instrument or device.

In a second step S2, a wireless data connection between a medical remote control and a controllable medical instrument or device is established by an initialization process.

In a third step S3, an input signal from the actuating button of the remote control is detected and forwarded to the control unit of the remote control.

In a fourth step S4, a control signal is determined by the control unit on the basis of the input signal and on the basis of a control state.

Finally, in a fifth step S5, the specific control signal is sent via the data interface of the remote control to the external data interface of the medical instrument or device in order to control it.

Finally, it should be noted that the features of the remote operator, the control system and the control method are also interchangeable.

1. A medical remote control for wirelessly controlling a medical instrument or a medical, digitally controllable device, the medical remote control comprising:
a housing having a fastening portion for fastening to an object;
an actuating button arranged in or on the housing for generating an input signal via manual actuation of the actuating button;
a wireless data interface for establishing a wireless data connection to an external data interface; and
a control unit adapted to process the input signal of the actuating button and to determine a control signal based on the input signal and to transmit the control signal via the wireless data interface,
the control unit being adapted to detect a temporal length and/or a number of actuations within a defined time interval of the actuating button as the input signal and to determine and transmit the control signal.

2. (canceled)

3. The medical remote control according to claim 1, wherein the control unit has a storage unit in which a current control state of the medical remote control is stored and the control unit is adapted to determine the control signal based on the current control state and the input signal.

4. The medical remote control according to claim 3, wherein a relation of control states is stored in the storage unit and the control unit proceeds to a subsequent, different control state corresponding to the relation of control states based on the relation of control states and an input signal selected for the relation of control states.

5. The medical remote control according to claim 3, wherein the medical remote control has a display, and the control unit is adapted to show the current control state of the medical remote control on the display.

6. The medical remote control according to claim 1, wherein the actuating button is adapted to have, a first degree of freedom for actuation that is translational and rectilinear, and a second degree of freedom about at least one pivot axis perpendicular to a longitudinal axis of the actuating button, and/or a third degree of freedom of rotation about the longitudinal axis of the actuating button in order to increase a number of actuation alternatives for input signals.

7. The medical remote control according to claim 1, wherein the fastening portion has an adhesive surface in order to firmly bond the medical remote control to the object.

8. The medical remote control according to claim 1, wherein the medical remote control is configured to be sterilizable and has a fluid-tight sheathing made of sterilizable materials.

9. The medical remote control according to claim 1, further comprising an inertial measurement unit to detect a spatial movement, the control unit being adapted to determine a corresponding control signal based on at least the input signal and the spatial movement.

10. A medical control system for controlling a medical instrument or a medical, digitally controllable device, the medical control system comprising:
at least one medical remote control according to claim 1; and
at least one digitally controllable medical instrument or device having an external data interface.

11. The medical control system according to claim 10, further comprising a central server with a server control unit, wherein the at least one digitally controllable medical instrument or device and the at least one remote control are connected to the central server as a client.

12. The medical control system according to claim 10, wherein the at least one digitally controllable medical instrument or device comprises a surgical robot with a robot arm, and wherein the medical remote control sends a translational movement command and/or a rotational movement command of the robot arm as the control signal in order to control and move the robot arm and an end effector attached to the robot arm.

13. The medical control system according to claim 10, wherein the at least one medical remote control comprises at least two medical remote controls that control the at least one digitally controllable medical instrument or device.

14. A medical control method for controlling a medical instrument or a medical electronic device using the medical remote control according to claim 1, the method comprising the steps of:
establishing a wireless data connection between the medical remote control and the medical instrument or the medical electronic device;
detecting an input signal of the actuating button of the remote control;
determining a control signal by the control unit based on the input signal; and
sending the control signal via the wireless data interface of the medical remote control to an external data interface of the medical instrument or the medical electronic device.

15. A computer-readable storage medium comprising instructions which, when executed by a computer, cause the computer to perform the medical control method of claim 14.
